(19) United States
(12) Patent Application Publication
(10) Pub. No.: US 2004/0092723 A1
(43) Pub. Date: May 13, 2004

(54) Compositions and methods for the intracellular delivery of antibodies

(76) Inventors: Bernard F. Erlanger, Whitestone, NY (US); Bi-Xing Chen, Fort Lee, NJ (US)

Correspondence Address: John P. White, Cooper & Dunham LLP, 1185 Avenue of the Americas, New York, NY, 10036, US

(21) Appl. No.: 10/618,179

(22) Filed: Jul. 11, 2003

Related U.S. Application Data
(60) Provisional application No. 60/395,363, filed on Jul. 11, 2002.
(60) Provisional application No. 60/471,113, filed on May 16, 2003.

(51) Int. Cl. C07K 16/46

(52) U.S. Cl. 530/391.1; 530/395

(57) ABSTRACT

This invention provides a composition of matter comprising an antibody and a peptide moiety, wherein the peptide moiety comprises an amino acid residue having a nitrogen-containing side chain and wherein the peptide is covalently bound to a carbohydrate moiety of the antibody. This invention also provides a pharmaceutical composition comprising the composition of matter and a pharmaceutically acceptable carrier. Finally, this invention provides methods and kits relating to the composition of matter.

This invention claims priority of U.S. Provisional Application No. 60/395,363, filed Jul. 11, 2002, and U.S. Provisional Application No. 60/471,113, filed May 16, 2003, the contents of which are hereby incorporated by reference into this application.

The invention described herein was made with government support under NIH Grant R01-HL47377. Accordingly, the United States government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

Antibodies are highly specific for their antigenic targets. This is particularly true of monoclonal antibodies. With certain individual exceptions, antibodies cannot penetrate extracellular membranes and enter cells. The exceptions, in most cases, are antibodies specific for nucleotides and ribonuclear proteins [1-3]. In one case the ability to enter cells could be associated with amino acid sequences in the variable region of the antibody [1]. The effectiveness of antibodies as research tools and as therapeutic agents would be considerably enhanced if they had the ability to enter cells so they could act intracellularly.

### SUMMARY OF THE INVENTION

This invention provides a composition of matter comprising an antibody and a peptide moiety, wherein the peptide moiety comprises an amino acid residue having a nitrogen-containing side chain and wherein the peptide is covalently bound to a carbohydrate moiety of the antibody.

This invention also provides six methods. The first method is for making the instant composition of matter comprising contacting an antibody with a peptide comprising an amino acid residue having a nitrogen-containing side chain under conditions permitting the peptide to covalently bind to a carbohydrate moiety of the antibody.

The second method is for introducing an antibody into a cell comprising contacting the cell with the instant composition of matter under conditions permitting entry of the composition into the cell, thereby introducing an antibody into the cell.

The third method is for determining whether an agent is present in a cell comprising (a) contacting the cell with an antibody that specifically forms a complex with the agent when contacted therewith, wherein (i) the antibody has a peptide covalently bound to a carbohydrate moiety of the antibody, the peptide comprising an amino acid residue having a nitrogen-containing side chain, and (ii) the contacting is performed under conditions permitting the antibody to enter the cell, and (b) determining whether such complex is present in the cell, the presence of such complex indicating that the agent is present in the cell.

The fourth method is for introducing an agent into a cell comprising contacting with the cell an antibody (i) having the agent affixed thereto and (ii) having a peptide moiety covalently bound to a carbohydrate moiety of the antibody, wherein the peptide moiety comprises an amino acid residue having a nitrogen-containing side chain, under conditions permitting the antibody to enter the cell, thereby introducing the agent into the cell.

The fifth method is for treating a subject afflicted with a disorder ameliorated by reducing the amount of, degrading, and/or interfering with the function of an intracellular agent in the subject's cells, which method comprises administering to the subject a therapeutically effective amount of an antibody, wherein (i) the antibody specifically binds to the intracellular agent when contacted therewith and (ii) the antibody has a peptide covalently bound to a carbohydrate moiety of the antibody, the peptide comprising an amino acid residue having a nitrogen-containing side chain, thereby treating the subject.

The sixth method is for treating a subject afflicted with a disorder ameliorated by the introduction of a therapeutic agent into the subject's cells, which method comprises administering to the subject a therapeutically effective amount of an antibody (i) having the agent affixed thereto and (ii) having a peptide moiety covalently bound to a carbohydrate moiety of the antibody, the peptide comprising an amino acid residue having a nitrogen-containing side chain, thereby treating the subject.

This invention further provides a pharmaceutical composition comprising the instant composition of matter and a pharmaceutically acceptable carrier.

Finally, this invention provides two kits. The first kit comprises the instant composition of matter and instructions for use. The second kit comprises the instant composition of matter and instructions for affixing an agent to the composition for delivery into a cell.

### BRIEF DESCRIPTION OF THE FIGURES

FIG. 1

This figure shows ELISA comparing anti-HIV-1 Gag activity with polyarginated anti-HIV-1 Gag. Concentrations of the antibody and the polyarginated antibody were the same (within 10%). Legend: [symbol] =antibody; [symbol] =polyarginated antibody.

FIGS. 2A & 2B

FIG. 2A shows the immunoperoxidase staining pattern of 3T3 cells exposed to YL1/2 after methanol fixation. FIG. 2B shows the pattern of 3T3 cells after exposure to polyarginated YL1/2.

FIGS. 3A, 3B & 3C

FIG. 3A shows anti-HIV-1 Gag vs. HeLa cells. FIG. 3B shows anti-HIV-1 Gag vs. P3×63.Ag8.653. FIG. 3C shows anti-HIV-Gag vs. a murine lung eridothelial cell line.

FIG. 4

Panel A: This panel shows anti-1d2 vs. SK-BR-3. Panel B: This panel shows anti-fullerene vs. HeLa cells. Panel C: This panel shows anti-3b3 vs. MCF-7 cells. Panel D: This panel shows anti-3b3 vs. SK-BR-3 cells.

FIGS. 5A & 5B

FIG. 5A shows the staining pattern of ob17 cells exposed to fluoresceinated, polyarginated anti-Gag: before methanol fixation. FIG. 5B shows the staining pattern of ob17 cells exposed to fluoresceinated, polyarginated anti-Gag: after methanol fixation.

### DETAILED DESCRIPTION OF THE INVENTION

### Definitions

As used in this application, except as otherwise expressly provided herein, each of the following terms shall have the meaning set forth below.

“Administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery.

“Affixed” shall mean attached by any means. In one embodiment, affixed means attached by a covalent bond. In another embodiment, affixed means attached non-covalently.

“Agent” shall mean any chemical entity, both organic or inorganic, including, without limitation, a glycomer, a protein, an antibody, a lectin, a nucleic acid, a small molecule, and any combination thereof. “Intracellular agent” shall include, without limitation, an enzyme, a metabolic or protein reactant or a metabolic or protein product.

“Amino acid,” “amino acid residue” and “residue” are used interchangeably herein to refer to an amino acid that is incorporated into a protein, polypeptide or peptide. The amino acid can be, for example, a naturally occurring amino acid or an analog of a natural amino acid that can function in a manner similar to that of the naturally occurring amino acid.

“Antibody” shall include, without limitation, (a) an immunoglobulin molecule comprising two heavy chains and two light chains and which recognizes an antigen; (b) a polyclonal or monoclonal immunoglobulin molecule; and (c) a monovalent or divalent fragment thereof. Immunoglobulin molecules may derive from any of the commonly known classes, including but not limited to IgA, secretory IgA, IgG, IgE and IgM. IgG subclasses are well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human or nonhuman. Nonhuman antibodies may be humanized by recombinant methods to reduce their immunogenicity in humans. Antibody fragments include, without limitation, Fab and F_{c} fragments, and antibodies having deleted therefrom a terminal portion of their F_{c} domain yet still possessing carbohydrate moieties.

“Humanized”, with respect to an antibody, means an antibody wherein some, most or all of the amino acids outside the CDR region are replaced with corresponding amino acids derived from a human immunoglobulin molecule. Small additions, deletions, insertions, substitutions or modifications of amino acids are permissible as long as they do not abrogate the ability of the antibody to bind a given antigen. Suitable human immunoglobulin molecules include, without limitation, IgG1, IgG2, IgG3, IgG4, IgA and IgM molecules. Various publications describe how to make humanized antibodies, e.g., U.S. Pat. Nos. 4,816,567, 5,225,539, 5,585,089 and 5,693,761, and PCT International Publication No. WO 90/07861.

“Conditions” permitting an antibody to enter a cell include, for example, physiological conditions.

“Detectable marker” includes, but is not limited to, a radioactive label, or a calorimetric, a luminescent, or a fluorescent marker. As used herein, such “labels” include radioactive isotopes, fluorescent groups and affinity moieties such as biotin that facilitate detection of the labeled peptide. Other labels and methods for attaching labels to compounds are well-known to those skilled in the art.

“Effective amount” means an amount sufficient to accomplish a specific task, e.g., treating a subject afflicted with a disorder. A person of ordinary skill in the art can perform routine titration experiments to determine such sufficient amount. “Therapeutically effective amount” shall mean an amount sufficient to treat a subject. The therapeutically effective amount of an agent will vary depending on the subject and upon the particular route of administration used. Based upon the agent, the amount can be delivered continuously, such as by continuous pump, or at periodic intervals (for example, on one or more separate occasions). Desired time intervals of multiple amounts of a particular agent can be determined without undue experimentation by one skilled in the art. In one embodiment, the effective amount of the instant composition is from about 1.0 ng/kg to about 100 mg/kg body weight of the subject. In another embodiment, the effective amount is from about 100 ng/kg to about 50 mg/kg body weight of the subject. In another embodiment, the effective amount is from about 1 μg/kg to about 10 mg/kg body weight of the subject. In a further embodiment, the effective amount is from about 100 μg/kg to about 1 mg/kg body weight of the subject.

“Moiety” shall mean, unless otherwise limited, any chemical or biochemical entity. Examples of moieties include, without limitation, proteins (antibodies), nucleic acids, carbohydrates, small molecules and inorganic compounds. In this invention, a carbohydrate moiety of an antibody means any carbohydrate found thereon including, without limitation, carbohydrate bound to the CH2 immunoglobulin domain.

The terms “nucleic acid”, “polynucleotide” and “nucleic acid sequence” are used interchangeably herein, and each refers to a polymer -of deoxyribonucleotides and/or ribonucleotides. The deoxyribonucleotides and ribonucleotides can be naturally occurring or synthetic analogues thereof. “Nucleic acid” shall mean any nucleic acid, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). Nucleic acids include, without limitation, anti-sense molecules and catalytic nucleic acid molecules such as ribozymes and DNAzymes. Nucleic acids also include nucleic acids coding for peptide analogs, fragments or derivatives which differ from the naturally-occurring forms in terms of the identity of one or more amino acid residues (deletion analogs containing less than all of the specified residues; substitution analogs wherein one or more residues are replaced by one or more residues; and addition analogs, wherein one or more resides are added to a terminal or medial portion of the peptide) which share some or all of the properties of the naturally-occurring forms.

“Pathogen” means an organism or virus capable of causing disease in animals, plants or microorganisms. Examples of pathogens include, without limitation, bacterial pathogens.

“Pharmaceutically acceptable carriers” include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. The carriers include but are not limited to an aerosol, intravenous, oral or topical carrier. Carriers are well known to those skilled in the art.

“Peptide” means a polymer of amino acid residues. The amino acid residues can be naturally occurring or chemical analogues thereof. Peptides include, but are not limited to, polypeptides and oligopeptides. Peptides can also include modifications such as glycosylation, lipid attachment, sulfation, hydroxylation, and ADP-ribosylation.

“Specifically bind” shall mean the binding of a first entity to a second entity based on complementarity between the three-dimensional structures of each. In one embodiment, specific binding occurs with a K_{D} of less than 10⁻⁸. In another embodiment, specific binding occurs with a K_{D} of less than 10⁻¹⁰. In a further embodiment, specific binding occurs with a K_{D} of less than 10⁻¹². As used herein, “specifically form a complex” shall be synonymous with “specifically bind”.

“Subject” shall mean any organism including, without limitation, a mouse, a rat, a dog, a guinea pig, a ferret, a rabbit and a primate. In the preferred embodiment, the subject is a human being.

“Toxin” means, without limitation, a poisonous substance produced by a cell (e.g., a substance encoded by a plasmid). Examples of toxins include, without limitation, endotoxins and exotoxins.

“Treating” means either slowing, stopping or reversing the progression of a disorder. As used herein, “treating” also means the amelioration of symptoms associated with the disorder.

### EMBODIMENTS OF THE INVENTION

This invention is based on applicants' surprising discovery that covalently linking poly-L-arginine to the oligosaccharide moiety of the CH2 region of an immunoglobulin makes possible penetration into the cytoplasm and, and in some cases into the nucleus of cells, without affecting specificity. Since the antibodies are covalently modified, they are suitable for use in intact animals.

Accordingly, this invention provides a composition of matter comprising an antibody and a peptide moiety, wherein the peptide moiety comprises an amino acid residue having a nitrogen-containing side chain and wherein the peptide is covalently bound to a carbohydrate moiety of the antibody.

In one embodiment of the instant composition of matter, the nitrogen-containing side chain comprises a guanido group. In a second embodiment, the peptide moiety comprises an amino acid residue selected from the group consisting of L-arginine, L-lysine and L-ornithine. In a third embodiment, the peptide moiety is selected from the group consisting of poly-L-arginine, poly-L-lysine and poly-L-ornithine.

In one embodiment, the peptide moiety has a molecular weight of between about 11 kD and about 16 kD. In another embodiment, the peptide moiety has a molecular weight of about 13 kD. In a further embodiment, the peptide moiety is ten or fewer amino acid residues in length. In one example, the peptide is an octapeptide. In another example, the peptide is HIV-Tat polypeptide having sequence gly-arg-lys-lys-arg-arg-gln-arg-arg-arg. In another embodiment, the peptide moiety is at least ten amino acid residues in length. In one example, the peptide moiety has a length of between about 10 amino acid residues and about 100 amino acid residues. In another example, the peptide moiety has a length of between about 25 amino acid residues and about 75 amino acid residues. In yet another example, the peptide moiety is about 68 amino acids in length.

In the instant composition, the antibody can be a monoclonal antibody or a polyclonal antibody.

In the instant composition of matter, the composition can be bound to a second moiety. In one example, the antibody and second moiety are covalently bound. In another example, the second moiety is selected from the group consisting of a detectable marker, a probe, a small molecule, a peptide, an antibody and a nucleic acid. Detectable markers include, for example, radioactive labels, and calorimetric, luminescent and fluorescent markers.

This invention provides six methods. The first method is for making the composition of matter comprising contacting an antibody with a peptide comprising an amino acid residue having a nitrogen-containing side chain under conditions permitting the peptide to covalently bind to a carbohydrate moiety of the antibody.

The second method is for introducing an antibody into a cell comprising contacting the cell with the composition of matter under conditions permitting entry of the composition into the cell, thereby introducing an antibody into the cell.

In one embodiment of the second method, the antibody alters a biochemical reaction in the cell by specifically binding to a reactant, a product or a catalyst of such reaction.

The third method is for determining whether an agent is present in a cell comprising (a) contacting the cell with an antibody that specifically forms a complex with the agent when contacted therewith, wherein (i) the antibody has a peptide covalently bound to a carbohydrate moiety of the antibody, the peptide comprising an amino acid residue having a nitrogen-containing side chain, and (ii) the contacting is performed under conditions permitting the antibody to enter the cell, and (b) determining whether such complex is present in the cell, the presence of such complex indicating that the agent is present in the cell.

In one embodiment of the third method, the antibody is labeled with a detectable marker.

The fourth method is for introducing an agent into a cell comprising contacting with the cell an antibody (i) having the agent affixed thereto and (ii) having a peptide moiety covalently bound to a carbohydrate moiety of the antibody, wherein the peptide moiety comprises an amino acid residue having a nitrogen-containing side chain, under conditions permitting the antibody to enter the cell, thereby introducing the agent into the cell.

In one embodiment of the fourth method, the agent is selected from the group consisting of a detectable marker, a probe, a small molecule, a peptide, an antibody and a nucleic acid.

The fifth method is for treating a subject afflicted with a disorder ameliorated by reducing the amount of, degrading, and/or interfering with the function of an intracellular agent in the subject's cells, which method comprises administering to the subject a therapeutically effective amount of an antibody, wherein (i) the antibody specifically binds to the intracellular agent when contacted therewith and (ii) the antibody has a peptide covalently bound to a carbohydrate moiety of the antibody, the peptide comprising an amino acid residue having a,nitrogen-containing side chain, thereby treating the subject.

The sixth method is for treating a subject afflicted with a disorder ameliorated by the introduction of a therapeutic agent into the subject's cells, which method comprises administering to the subject a therapeutically effective amount of an antibody (i) having the agent affixed thereto and (ii) having a peptide moiety covalently bound to a carbohydrate moiety of the antibody, the peptide comprising an amino acid residue having a nitrogen-containing side chain, thereby treating the subject.

In one embodiment of the fifth and sixth methods, the subject is human.

In the fifth and sixth methods, the disorder can be associated with (a) the presence of a toxin in the subject; (b) cancer; or (c) the presence of a pathogen in the subject. In one example, the disorder is caused by the HIV virus.

This invention provides a pharmaceutical composition comprising the instant composition of matter and a pharmaceutically acceptable carrier.

Finally, this invention provides two kits: The first kit comprises the instant composition of matter and instructions for use. The second kit comprises the instant composition of matter and instructions for affixing an agent to the composition for delivery into a cell. The second kit can further comprise reagents for affixing the agent to, the composition.

This invention will be better understood from the Experimental Details that follow. However, one skilled in the art will readily appreciate that the specific methods and results discussed are merely illustrative of the invention as described more fully in the claims which follow thereafter.

### Experimental Details

We report a general procedure for chemical modification of immunoglobulins that makes possible their entrance into cells, without affecting specificity. The monoclonal antibodies studied are of the IgG isotype. The strategy exploits the fact that immunoglobulins have oligosaccharide sequences covalently linked to asparagine 297 in the CH2 domain [4]. The presence of this structure has no effect on the binding specificity of the antibody and has been used by others to attach radioactive and anti-tumor agents by periodate oxidation of the oligosaccharide and then linkage of an agent via Schiff base formation [5,6].

Here we report the linkage of poly-L-arginine to monoclonal antibodies of different specificities and their subsequent ability to penetrate into different cell lines. Retention of specificity is demonstrated by ELISA with an anti-HIV Gag antibody and intracellularly with a monoclonal anti-tubulin antibody.

### I. Materials and Methods

### A. Cell Lines

A murine lung endothelial cell line, HeLa and 3T3 cell lines were used. P3×63Ag.8.653 is a non-producing myeloma line used as a fusion partner for the preparation of mouse hybridomas. SK-Br-3 (ATCC, HTB-30) and MCF-7 (ATCC, HTB-22) are breast cancer lines. The ob17 cells, a preadipocyte cell line, were described previously [7].

### B. Monoclonal Antibodies

The anti-fullerene antibody has been described before [8,9]. The anti-HIV-1 Gag antibody is p24-specific and was produced by a cell line obtained from NIH. It is listed in the NIH/AIDS Research and Reference Reagent Program catalog as line 183-H12-5C (Catalog no. 1513). Two mouse anti-tumor IgG antibodies, 1d2 and 3b3, were used. The anti-tubulin antibody, YL1/2 [10], was also used.

### C. Reactions with poly-L-arginine

We illustrate the coupling procedure using the anti-HIV-1 Gag antibody. The other antibodies were linked to poly-L-arginine by the identical procedure.

To 0.5 ml of anti-HIV-1 Gag containing 0.5 mg of antibody in PBS was added 4 mg of sodium periodate dissolved in 50 μl of distilled water. (The periodate solution was prepared just prior to addition to the antibody). The reaction solution was allowed to stand at room temperature for one hour. It was then dialyzed for 2-3 hours against a solution of 0.1N NaHCO₃ containing 0.5N NaCl that had been adjusted to pH 9.5. To this solution was added 50 μl of a solution of 0.3 mg of poly-L-arginine hydrochloride (Sigma, P4663 Molecular weight 8,500-13,000, average number of Arg residues: 68) dissolved in 150 μl of distilled water. (The molar ratio of poly-L-arginine to antibody was about 3 to 1). The solution remained clear. It was left overnight at room temperature, after which 4 mg of NaBH₄ dissolved in a minimal amount of cold distilled water was added and the reaction mixture was allowed to stand at room temperature for two hours, followed by dialysis against three changes of PBS containing 0.5N NaCl, using a dialysis membrane with a cut-off of molecular weight 50,000. The final solution was then, if necessary, clarified by centrifugation and the supernatant tested for penetration into various cell lines.

### D. Fluorescein-Labeled Polyarginated anti-HIV-1 Gag

The polyarginated antibody (0.5 mg/ml in PBS) was dialyzed for three hours against 0.1N NaHCO₃, adjusted to pH 8.5. To this solution was added 15 μl of a 1 mg/ml solution of 5(and 6) carboxyfluorescein succinimidyl ester (Molecular Probes, Eugene, Oreg.) in dry pyridine. The reaction mixture was rocked overnight at room temperature and then dialyzed against three changes of PBS.

### E. Retention of Specificity by Polyarginated anti-HIV-1 Gag

An ELISA was carried out on anti-HIV Gag before and after polyargination, as follows: wells in a 96 well polystyrene plate (Corning) were coated with a solution of HIV-1 Gag (1 μg/ml) in 0.1N NaHCO₃. Then serial dilutions of anti-HIV Gag or the polyarginated antibody in PBS-0.1% Tween 20 were added to the wells. Incubation was for two hours at 37° C. After washing the wells three times with PBS-0.1% Tween 20, peroxidase-labeled goat anti-mouse IgG in PBS-Tween 20 was added to the wells, followed by a one hour incubation. After three washes with PBS-0.1% Tween 20, color development was with o-phenylenediamine (Sigma) with color measurement at 490 nm.

Retention of specificity was also confirmed by immunohistochemistry using anti-tubulin antibody YL1/2 [10]. This was accomplished by first studying the pattern of immunoperoxidase staining 3T3 cells after the conventional histochemical technique, i.e. methanol fixation of the cells followed by exposure to specific antibody (i.e, YL1/2) and visualizing by the immunoperoxidase technique. The staining pattern was then compared to that obtained using the polyarginated antibody, prior to fixation, as described below. As YL1/2 is a rat antibody, the second antibody used in both procedures was peroxidase-labeled goat F(Ab′)₂ anti-rat IgG (Biosource AR1440-4).

### F. Preparation of Slides for Study of Intracellular Antibody

The HeLa, P3, and the murine lung endothelium cells were grown in Dulbecco's Modified Eagle Medium (DMEM) (GibcoBRL) supplemented with 10% fetal calf serum. The SK-BR-3 line was grown in McCoy's 5a media (Cellgro, 10-050-CV) supplemented with 200 mM L-glutamine, 100 mM sodium pyruvate, vitamins (Cellgro, 25-020-CI) and 10% heat inactivated bovine serum. The MCF-7 line was grown in MEM (Cellgro, 10-010-CV) supplemented similarly. All cell lines were grown in Lab-Tek Chamber slides (Nalge Nunc International, Naperville, Ill.). After incubation at 37° C. for 2-3 days, the medium was replaced with 200 μl of medium containing 30 μl of unmodified (control) or polyarginated antibody. The chamber slides were then incubated at 37° C. for four hours, washed with PBS and the cells fixed with methanol at −20° C. for one minute. This was followed by drying in air and immersion in a PBS solution to rehydrate the cells. The PBS was replaced by 200 μl of DMEM containing 20% fetal calf serum and 4 μl of peroxidase-labeled goat anti-mouse IgG (Sigma). Incubation at room temperature for one hour on a slow moving shaking platform was followed by three washes with PBS-Tween 20, decanting, addition of 250 μl of the peroxidase substrate (Pierce Cat# 299067) and development for 10 to 15 minutes. After washing three times with PBS-Tween 20 and rinsing with distilled water, the chamber fittings were removed and replaced by cover slips for microscopic examination.

### II. Results

### A. Effect of Antibody Modification by Polyarginine on Specificity of Binding

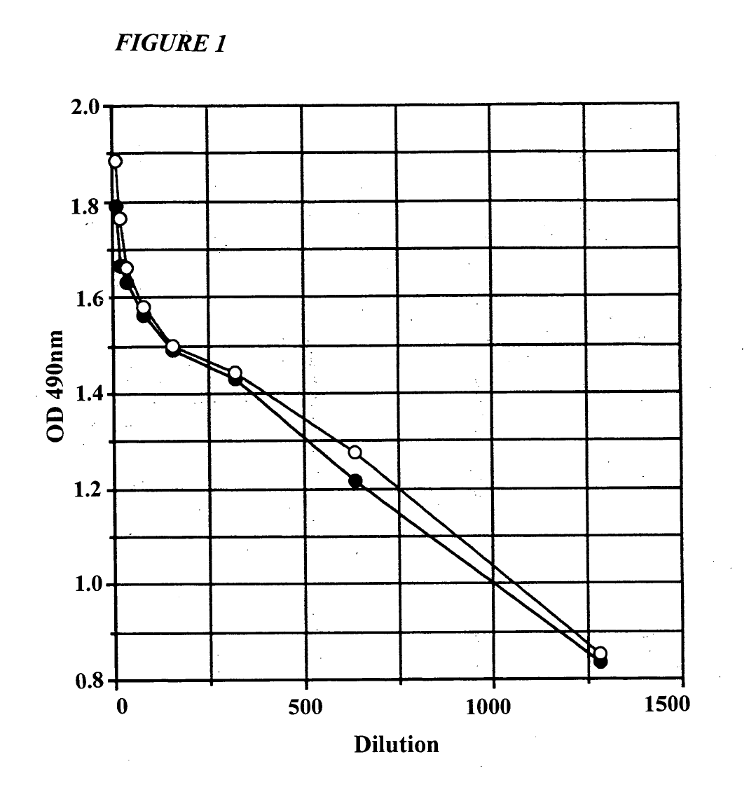

It was important to show that modification of antibody by covalent attachment of polyarginine did not affect specificity, although in earlier studies on chemical modification of the oligosaccharide region of IgG, no change occurred [5,6]. FIG. 1 demonstrates retention of specificity by anti-Gag, as determined by ELISA. Similarly, anti-fullerene was unaffected by covalent binding of polyarginine (not shown). As a control for specificity, the antibodies did not bind to bovine serum albumin.

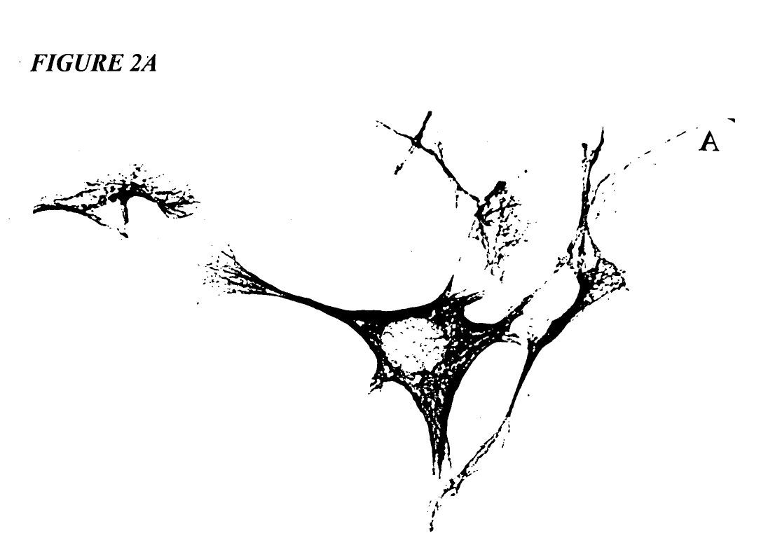

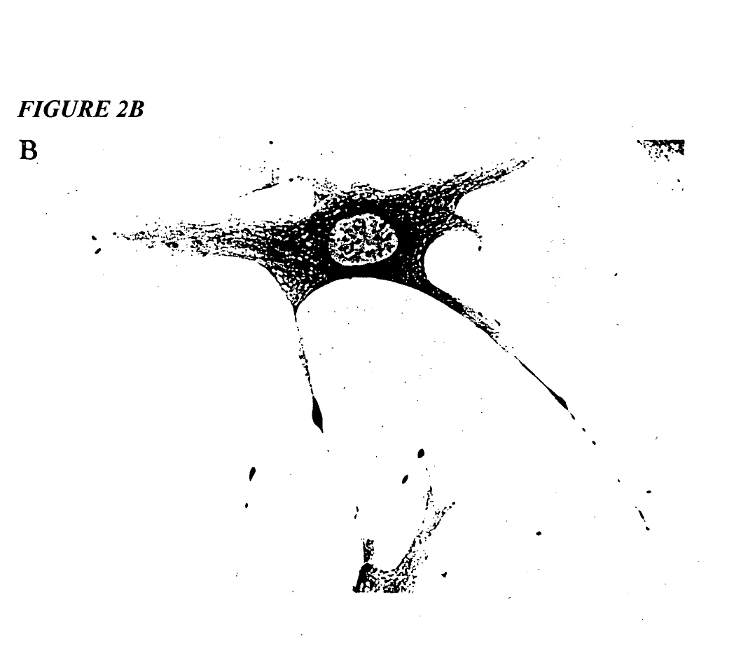

Retention of specificity was also shown immunohistochemically using an anti-tubulin antibody [10] and 3T3 cells. FIG. 2A shows the staining pattern obtained using the traditional staining procedure, i.e., methanol fixation followed by exposure to anti-tubulin antibody, YL1/2. FIG. 2B shows the pattern obtained with polyarginated YL1/2. Specific staining of microtubules is apparent in both preparations.

### B. Intracellular Delivery of Monoclonal Anti-Gag Antibody

All of the antibodies were tested on all cell lines for cellular penetration prior to modification. With one exception, 3B3, no staining was seen. The fields were essentially blank, i.e., not even surface binding could be seen. With respect to unmodified 3B3, faint staining of cell membranes and cytoplasm could be seen.

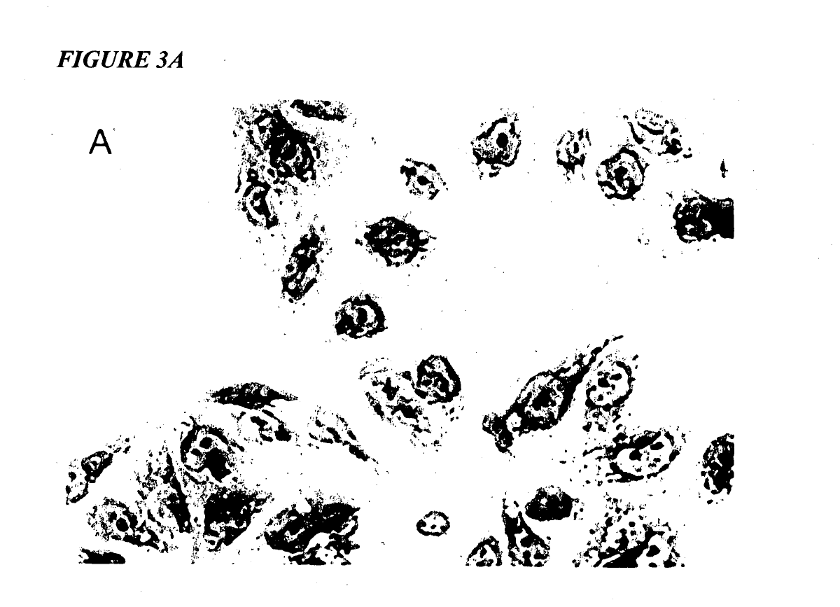

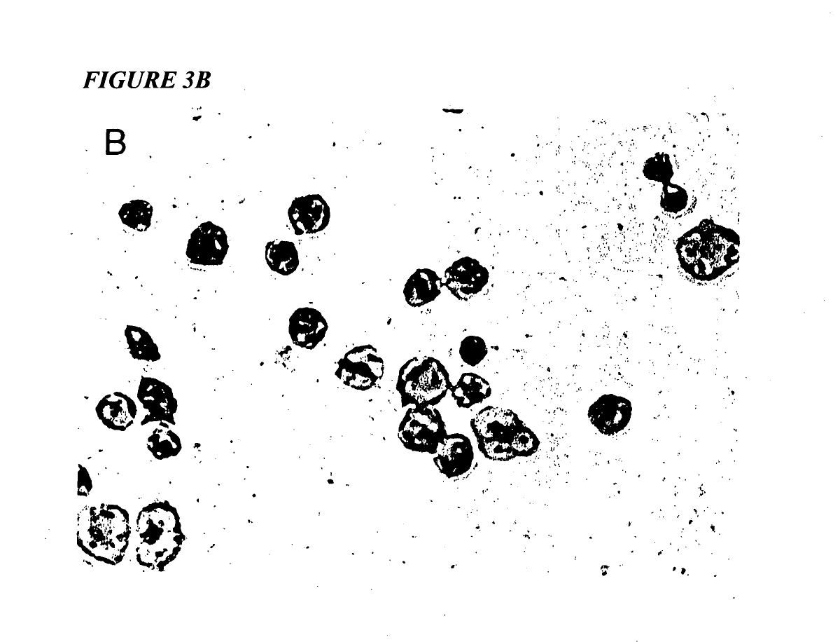

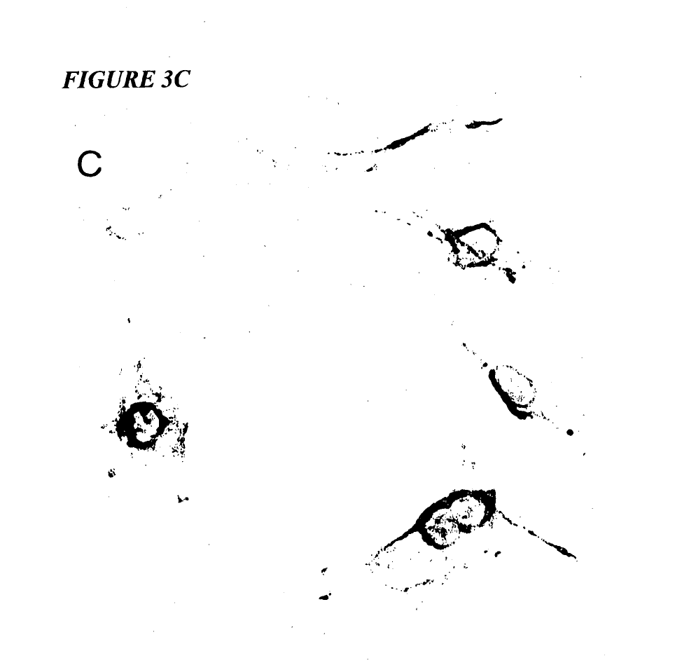

The first studies were carried out with the monoclonal anti-HIV-1-Gag antibody. Three cell lines were examined for intracellular penetration: HeLa, P3×63-Ag.8.653 (a mouse myeloma) and the murine lung endothelial cell line. Antibody was seen in the cytoplasm and nuclei of the HeLa and mouse endothelial cell lines (FIGS. 3A and 3C) Antibody was seen only in the nucleus of the mouse myeloma line; apparently all had migrated there from the cytoplasm (FIG. 3B).

### C. Intracellular Delivery of Other Antibodies

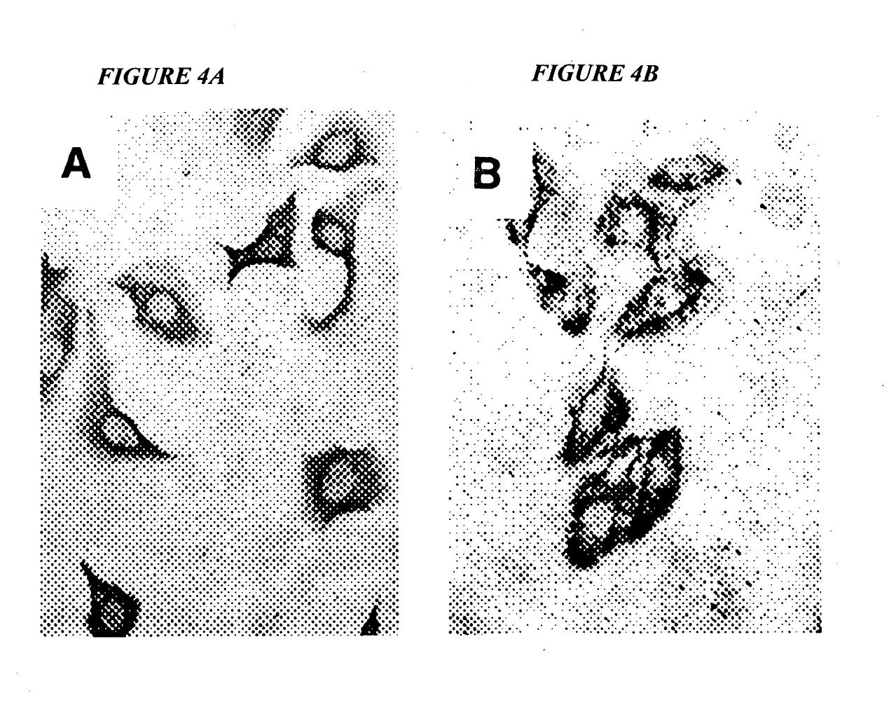

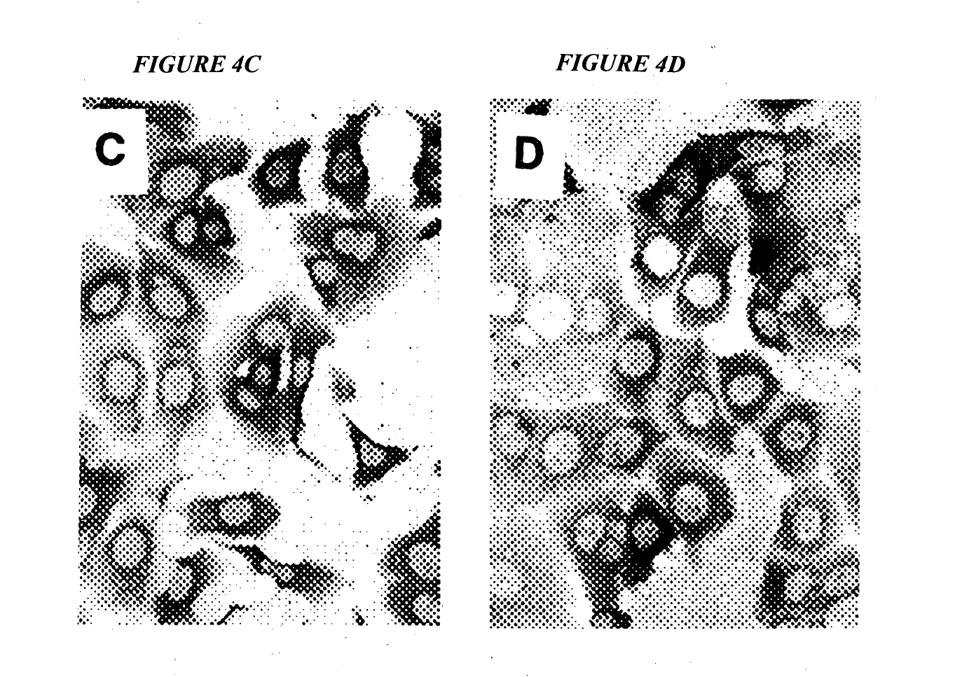

Three antibodies were examined: an anti-fullerene [8,9], and two anti-tumor antibodies, 1d2 and 3b3 (see experimental protocol). In all cases (FIG. 4) strong cytoplasmic staining was seen. Any nuclear staining was weak. In the case of the anti-fullerene antibody small bodies of precipitate were seen in the cytoplasm as if the antibody cross reacted with some cytoplasmic component.

### D. Eliminating Methanol Fixation as Cause of Intracellular Delivery

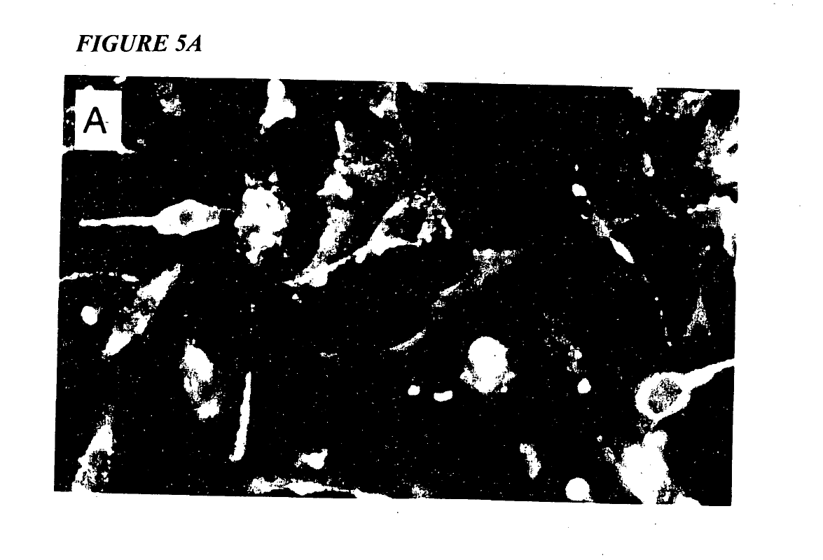

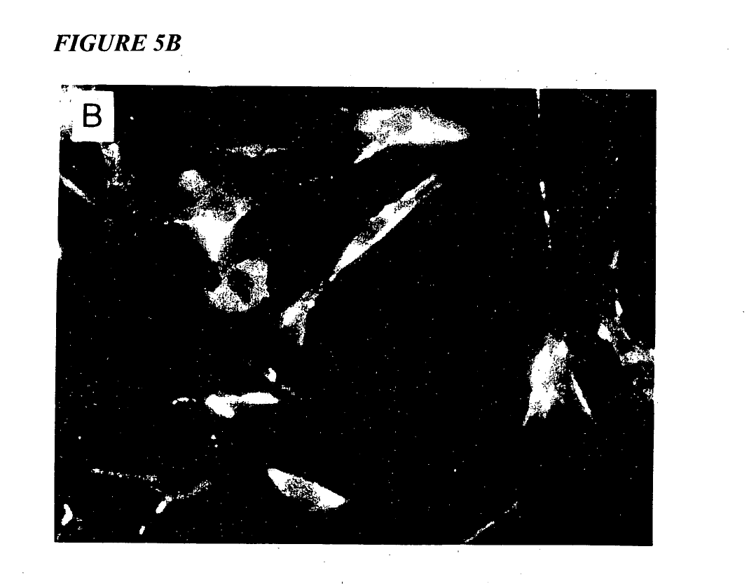

Lundberg and Johannson have shown that methanol fixation can cause artificial import of a protein into cells [11]. In order to eliminate that possibility, we compared the staining of ob17 cells with fluoresceinated polyarginated anti-Gag before and after methanol fixation (FIGS. 5A and 5B). The fluorescence patterns are essentially identical.

### III. Discussion

We have demonstrated that covalent attachment of poly-L-arginine to the oligosaccharide of immunoglobulins converts them into reliable intracellular reagents. Our success with this arginine homopolymer includes arginine-containing oligopeptides related to those described in the literature as capable of acting as intracellular carriers. Among them are HIV-1 Tat sequences which have been used by others to carry normally extracellular entities into cells, examples being beta-galactosidase, horse radish peroxidase, ribonuclease A, pseudomonas exotoxin and magnetic nanoparticles [12,13]. We had success with the HIV-Tat peptide sequence, gly-arg-lys-lys-arg-arg-gln-arg-arg-arg, linked to the oligosacharride of IgG.

With respect to oligopeptides of arginine, it is reported that linking hepta-arginine to cyclosporin A makes possible intradermal delivery of the drug [14]. Oligoarginines of varying lengths joined to fluorescein have been shown to enter a number of cell lines, with maximal uptake occurring with Arg15 (MW ca. 2,600) and activity decreasing markedly with increasing size [15,16,17]. We found that using our method octa-arginine was effective as an intracellular transporter of immunoglobulins.

Conversion of monoclonal antibodies into reliable intracellular agents extends their already significant applications. As research tools, they have the potential to be delivered intracellularly to investigate the functions of organelles, enzymes, structural elements and other intracellular entities in living cells. Finally, as the antibodies are covalently modified, they have the potential to be used in intact animals. In an intact animal, they would be diluted out to the extent that they would select their targets according to their specificities for particular cell surface receptors. For example; they could be targeted to specific membrane-linked tumor antigens to deliver toxic agents intracellularly.

### References

[1] A. Avrameas, T. Ternynck, F. Nato, G. Buttin, S.

Avrameas (1998) Proc. Natl. Acad. Sci. USA 95: 5601-5608.

[2] E. Koren, M. Koscec, M. Wolfson-Reichlin, F. M.

Ebling, B. Tsao, M. B. H. Hahn, M. Reichlin (1995) J. Immunol. 154: 4857-4864.

[3] J. Ma, J. M. G. V. Chapman, S. L. Chen, G. Melick, R. Penny, S. N. Breit (1991) Clin. Exp. Immunol. 84: 83-91.

[4] B. J. Sutton, D. C. Phillips (1983) Biochem. Soc. Trans. 11: 130-132.

[5] J. D. Rodwell, V. L. Alvarez, C. Lee, L. D. Lopes, J. W. F. Goers, H. D. King, H. J. Powsner, T. J. McKearn (1986) Proc. Natl Acad. Sci. USA 83: 2632-2636.

[6] M. Awwad, P. G. Strome, S. C. Gilman, H. R. Axelrod (1994) Cancer Immunol. Immunother. 38: 23-30.

[7] I. Tatsis-Kotsidis, B. F. Erlanger (1999) Biochem. Pharmacol. 58: 168-170.

[8] B. -X. Chen, S. R. Wilson, M. Das, D. J. Coughlin, B. F. Erlanger (1998) Proc. Natl. Acad. Sci USA 95: 10809-10813.

[9] B. C. Braden, F. A. Goldbaum, B. -X. Chen, A. N. Kirschner, S. R. Wilson, B. F. Erlanger (2000) Proc. Natl. Acad. Sci. USA 97: 12193-12197.

[10] J. V. Kilmartin, B. Wright, C. Milstein (1982) J. Cell Biol. 93: 576-582.

[11] M. Lundberg, M. Johansson (2001) Nature Biotechnology 19: 713.

[12] S. Fawell, J. Seery, Y. Daikh, C. Moore, L. L. Chen, B. Pepinsky, J. Barsoun (1994) Proc. Natl. Acad. Sci. USA 91: 664-668.

[13] M. Lewin, N. Carlesso, C. -H. Tung, X. -W. Tang, D. T. Scadden, R. Weissleder (2000) Nature Biotechnology 118: 410-414.

[14] J. B. Rothbard, S. Garlington, Q. Lin, T. Kirschberg, E. Kreider, P. L. McGrane, P. A. Khavari (2000) Nature Medicine 6: 1253-1257.

[15] D. J. Mitchel, D. T. Kim, L. Steinman, C. G. Fathman, J. B. Rothbard (2000) J. Peptide Res. 56: 318-325.

[16] P. A. Wender, D. J. Mitchell, K. Pattabiraman, E. T. Peelkey, L. Steinman, J. B. Rothbard (2000) Proc. Natl. Acad. Sci. USA 97: 13003-13008.

[17] M. Buschle, W. Schmidt, W. Zauner, K. Mechtler, B. Trska, H. Kirlappos, M. Birnsteil (1997) Proc. Natl Acad. Sci. USA 94: 3256-3261.

What is claimed is:

1. A composition of matter comprising an antibody and a peptide moiety, wherein the peptide moiety comprises an amino acid residue having a nitrogen-containing side chain and wherein the peptide is covalently bound to a carbohydrate moiety of the antibody.

2. The composition of claim 1, wherein the nitrogen-containing side chain comprises a guanido group.

3. The composition of claim 1, wherein the peptide moiety comprises an amino acid residue selected from the group consisting of L-arginine, L-lysine and Lornithine.

4. The composition of claim 1, wherein the peptide moiety is selected from the group consisting of poly-L-arginine, poly-L-lysine and poly-L-ornithine.

5. The composition of claim 1, wherein the peptide moiety comprises poly-L-arginine.

6. The composition of claim 1, wherein the peptide moiety comprises poly-L-lysine.

7. The composition of claim 1, wherein the peptide moiety comprises and poly-L-ornithine.

8. The composition of claim 1, wherein the peptide moiety has a molecular weight of between about 11 kD and about 16 kD.

9. The composition of claim 8, wherein the peptide moiety has a molecular weight of about 13 kD.

10. The composition of claim 1, wherein the peptide moiety is ten or fewer amino acid residues in length.

11. The composition of claim 10, wherein the peptide is an octa-peptide.

12. The composition of claim 10, wherein the peptide moiety is HIV-Tat polypeptide having sequence gly-arg-lys-lys-arg-arg-gln-arg-arg-arg.

13. The composition of claim 1, wherein the peptide moiety is at least ten amino acid residues in length.

14. The composition of claim 13, wherein the peptide moiety has a length of between about 10 amino acid residues and about 100 amino acid residues.

15. The composition of claim 14, wherein the peptide moiety has a length of between about 25 amino acid residues and about 75 amino acid residues.

16. The composition of claim 15, wherein the peptide moiety is about 68 amino acids in length.

17. The composition of claim 1, wherein the antibody is a monoclonal antibody.

18. The composition of claim 1, wherein the antibody is a polyclonal antibody.

19. The composition of claim 1, wherein the composition is bound to a second moiety.

20. The composition of claim 18, wherein the antibody and second moiety are covalently bound.

21. The composition of claim 19, wherein the second moiety is 'selected from the group consisting of a detectable marker, a probe, a small molecule, a peptide, an antibody and a nucleic acid.

22. The composition of claim 21, wherein the detectable marker is selected from the group consisting of a radioactive label, and a calorimetric, luminescent or fluorescent marker.

23. A method for making the composition of claim 1 comprising contacting an antibody with a peptide comprising an amino acid residue having a nitrogen-containing side chain under conditions permitting the peptide to covalently bind to a carbohydrate moiety of the antibody.

24. A method for introducing an antibody into a cell comprising contacting the cell with the composition of claim 1 under conditions permitting entry of the composition into the cell, thereby introducing an antibody into the cell.

25. The method of claim 24, wherein the antibody alters a biochemical reaction in the cell by specifically binding to a reactant, a product or a catalyst of such reaction.

26. A method for determining whether an agent is present in a cell comprising (a) contacting the cell with an antibody that specifically forms a complex with the agent when contacted therewith, wherein (i) the antibody has a peptide covalently bound to a carbohydrate moiety of the antibody, the peptide comprising an amino acid residue having a nitrogen-containing side chain, and (ii) the contacting is performed under conditions permitting the antibody to enter the cell, and (b) determining whether such complex is present in the cell, the presence of such complex indicating that the agent is present in the cell.

27. The method of claim 26, wherein the antibody is labeled with a detectable marker.

28. A method for introducing an agent into a cell comprising contacting with the cell an antibody (i) having the agent affixed thereto and (ii) having a peptide moiety covalently bound to a carbohydrate moiety of the antibody, wherein the peptide moiety comprises an amino acid residue having a nitrogen-containing side chain, under conditions permitting the antibody to enter the cell, thereby introducing the agent into the cell.

29. The method of claim 28, wherein the agent is selected from the group consisting of a detectable marker, a probe, a small molecule, a peptide, an antibody and a nucleic acid.

30. A method for treating a subject afflicted with a disorder ameliorated by reducing the amount of, degrading, and/or interfering with the function of an intracellular agent in the subject's cells, which method comprises administering to the subject a therapeutically effective amount of an antibody, wherein (i) the antibody specifically binds to the intracellular agent when contacted therewith and (ii) the antibody has a peptide covalently bound to a carbohydrate moiety of the antibody, the peptide comprising an amino acid residue having a nitrogen-containing side chain, thereby treating the subject.

31. A method for treating a subject afflicted with a disorder ameliorated by the introduction of a therapeutic agent into the subject's cells, which method comprises administering to the subject a therapeutically effective amount of an antibody (i) having the agent affixed thereto and (ii) having a peptide moiety covalently bound to a carbohydrate moiety of the antibody, the peptide comprising an amino acid residue having a nitrogen-containing side chain, thereby treating the subject.

32. The method of claim 30 or 31, wherein the subject is human.

33. The method of claim 30 or 31, wherein the disorder is associated with the presence of a toxin in the subject.

34. The method of claim 30 or 31, wherein the disorder is cancer.

35. The method of claim 30 or 31, wherein the disorder is associated with the presence of a pathogen in the subject.

36. The method of claim 35, wherein the disorder is caused by the HIV virus.

37. A pharmaceutical composition comprising the composition of claim 1 and a pharmaceutically acceptable carrier.

38. A kit comprising the composition of claim 1 and instructions for use.

39. A kit comprising the composition of claim 1 and instructions for affixing an agent to the composition for delivery into a cell.

40. The kit of claim 39, further comprising reagents for affixing the agent to the composition.
